(19) United States
(12) Patent Application Publication
(10) Pub. No.: US 2004/0215118 A1
(43) Pub. Date: Oct. 28, 2004

(54) Disposable, water resistant surgical bandage or cast cover with powder dispensing rings at any opening

(76) Inventors: Cynthia Dumas, Dallas, TX (US); Richard S. Dietz, Chicago, IL (US)

Correspondence Address: LITMAN LAW OFFICES, LTD., P.O. BOX 15035 CRYSTAL CITY STATION, ARLINGTON, VA, 22215, US

(21) Appl. No.: 10/423,867

(22) Filed: Apr. 28, 2003

(51) Int. Cl. A61F 13/00

(52) U.S. Cl. 602/3; 602/60

(57) ABSTRACT

Disposable, water resistant plastic surgical bandage or cast covers with elastic bands at any open end, and powder rings inside at all open ends that gel on contact with any moisture. The covers are patterned for the head, neck, chest, waist, arm, leg, and foot to protect against water seepage when taking a shower or bath.

### BACKGROUND OF THE INVENTION

1. Field of the Invention

The present invention relates generally to water resistant coverings. More specifically, the invention is a disposable water repellant, plastic, surgical bandage or cast covering having a waterproof sleeve with an elastic band on any open end. An absorbent powder ring distributed within a wicking material inside the sleeve adjacent an open end when moistened would form a waterproofing sealing gel barrier. The invention is adapted to different sizes and shapes relative to the specific body part to be protected from water while taking a shower or bath. This waterproofing protection will be continued with each wetting until the injury has healed.

2. Description of the Related Art

The prior art of interest descries various waterproofing covers for injured areas of a body, but none discloses the present invention. There is an urgent need for an economical disposable waterproof body part cover that will enable the injured person or animal to shower or take a bath with the bandage or cast on while the injury heals. The related art of interest will be discussed in the order of perceived importance relative to the present invention.

U.S. Pat. No. 6,191,335 issued on Feb. 20, 2001, to Joseph W. Robinson describes a wound dressing consisting of a backing layer of hydrophilic polyurethane, an apertured or net wound facing layer of polyurethane, and an intermediate absorbent layer comprising a cotton fiber support layer containing a polyacrylic acid based superabsorber material. The absorbent fiber material will form a gel upon contact with any moisture. The device is distinguishable for requiring cotton fibers and polyacrylic acid based superabsorber material backed by a hydrophilic polyurethane layer.

U.S. Pat. No. 5,715,841 issued on Feb. 10, 1998, to Leo J. Utecht describes a personal protection apparatus with an adhesive useful for covering burn areas, wound areas and contaminated surfaces of various kinds. The barrier comprises layers of absorbent material that may be a gel, hydrogel, hydrophobic web, for natural or synthetic fibrous material. The devices are distinguishable for requiring various gels, hydrogels, hydrophobic webs, or synthetic fibrous materials.

U.S. Pat. No. 6,512,158 B1 issued on Jan. 28, 2003, to John A. Dobos describes a medical protective wrap device comprising a rectangular liquid impermeable and stretchable polymeric wrap that provides a watertight seal and barrier for wounds and seepage.

Sealing cuffs are provided on opposite edges with reduced stretchability relative to the sheet. A fastener is attached to the first terminal edge to secure the sheet to the body. The second terminal edge is fastened to the sheet after at least one wrapping around the body part. The devices are distinguishable for requiring a polymeric wrap without gelling seals.

U.S. Patent Application NO. 2002/0091347 A1 published on Jul. 11, 2002, for Thomas G. Eakin describes a wound dressing composition comprising an inner covering release layer consisting of paper coated with either polyethylene or silicon, a dressing layer comprising a gelling layer of either carboxymethylcellulose alone with starch or sticky polyisobutylene (optionally mixed with polyethylene), and an indicator layer comprising dots of a soluble dye mixed with sodium carboxymethyl cellulose or polyisobutylene. Moisture from the wound would cause the activation of the indicator layer. The wound dressing composition is distinguishable for requiring activation of dyes by leaking moisture.

U.S. Patent Application No. 2002/0156411 A1 published on Oct. 24, 2002, for Helge Ahrens et al. describes a dressing composition comprising a top layer of a transparent multilayered polyurethane film over an adhesive layer comprising a polyacrylate including a hydrocarb on tackifier added to enhance adhesion to the skin. The dressing is beveled from its center. The dressing composition is distinguishable for requiring a beveled shape, a multilayered polyurethane film, and an adhesive layer of a polyacrylate and a hydrocarbon tackifier.

U.S. Pat. No. 4,523,586 issued on Jun. 18, 1985, to Mark S. Couri describes a protective cover for a limb or a cast comprising a flexible fabric bag having hook and loop fastening patches or snaps to close the bag about a limb tightly. The covers are distinguishable for requiring only fabric bags and fastening.

U.S. Pat. No. 5,048,122 issued on Sep. 17, 1991, to Deborah G. Prieur describes a baby garment or creeper for shielding lines such as catheters to the infant patient comprising a close fitting soft cotton garment body having a zippered front portion. The garment is distinguishable for lacking waterproofing.

U.S. Pat. No. 5,063,919 issdued on Nov. 12, 1991, to Doris C. Silverberg describes a waterproof protective sleeve made of polyethylene-vinyl for protecting casts, splints or other surgical dressings on arms, legs, and individual fingers from water damage comprising a combination of a securing tubular polyethylene-vinyl bag member and numerous encircling straps, a non-skid end strip to secure the bag member with the elastic members, and to provide a waterproof seal between the member and the wearer. The devices are distinguishable for requiring numerous encircling straps, end strips, and fingers.

U.S. Pat. No. 5,152,741 issued on Oct. 6, 1992, to Frank G. Farnio describes a surgical female chest or bra dressing comprising a flexible band formed primarily of a stretchable material with free overlapping ends that engage between the breasts. The band includes differential support structure to provide more support for the sides of the user. The surgical chest dressing is distinguishable for its required bra structure and the lack of a plastic waterproof cover.

U.S. Pat. No. 5,257,956 issued on Nov. 2, 1993, to Carol J. Ewen describes a post-mastectomy garment comprising a padded vest-like garment adapted for applying comforting pressure to the sites of breast removal and other tissues, and for holding pain relieving packages. The garment is distinguishable for being limited to a vest adapted to hold packages.

U.S. Pat. No. 5,395,302 issued on Mar. 7, 1995, to Rudolph P. Botha et al. describes a protective waterproof sheath for a bandaged injured limb or in a cast comprising a polyvinyl bag made by sealing two sheets to fit a hand and forearm. The sheath can have both ends open to fit an elbow or a knee. The end has a closing strap that is more plasticized than the bag, and attached with hook and loop fastening patches. The device is distinguishable for requiring straps to prevent wetting of the bandaged region and the lack of the inventive gelling seals.

U.S. Pat. No. 5,562,642 issued on Oct. 8, 1996, to James A. Smith et al. describes separately packaged applicator pads for topical delivery of two incompatible dermatological drugs comprising a plurality of rectangular compartmentalized applicator pads that may be exposed and sequentially applied to the afflicted skin area. The device is distinguishable for requiring a plurality of compartmentalized applicator pads.

U.S. Pat. No. 6,503,526 B1 issued on Jan. 7, 2003, to Duane G. Krzysik et al. describes absorbent article compositions containing natural fats, natural oils, sterols or sterol derivatives, emollients, solidifying agents, and viscosity enhancers. The absorbent article comprises an outer cover, a liquid permeable bodyside liner, an absorbent body, and the chemical composition. The articles are distinguishable for requiring the addition of a specific dissimilar chemical composition.

U.S. Pat. No. 6,511,467 B1 issued on Jan. 28, 2003, to Denise Reuss describes a fabric drainage reservoir medical garment including one or more drainage reservoirs in pockets. The medical garments are distinguishable for requiring pockets to hold drainage reservoirs.

European Patent Application No. EP 1 184 039 A2 published on Mar. 6, 2002d, for Yoshihide Murakami et al. describes an adhesive tape or sheet for application skin made of an acrylic copolymer having a specific carboxylic acid ester, wherein the acrylic copolymer has a gel fraction adjusted to a specific range. The adhesive tape or sheet can be used for first aid adhesive plasters, surgical tapes, large adhesive plasters with a pad, dressing materials, and the like. The device is distinguishable for being limited to an adhesive tape or sheet requiring acrylic copolymers.

None of the above inventions and patents, taken either singly or in combination, is seen to describe the instant invention as claimed. Thus, a disposable, water resistant surgical bandage or cast cover with internal powder dispensing rings at any open end solving the aforementioned problems is desired.

### SUMMARY OF THE INVENTION

The present invention is directed to an economical and ergonomic disposable water resistant surgical bandage or cast covering on various parts of a human body that is disposable, and waterproofs the bandage or cast when the injured person desires to take a shower or bath. For a waterproof cover adapted to cover a bandaged arm or leg portion, a plastic sheath having elastic bands on both ends affirmatively closes the ends on the body portion. Inside the sheath and proximate to its ends are provided powdered polymeric rings that gel when wet to effectively seal the sheath from encroaching moisture and water. The invention is adaptable to fit various parts of the human body such as the head, neck, upper arm, forearm, hand, chest back, waist, buttocks, thigh, calf, and foot. In the instances of covering a hand or a foot cast, the sheath can be limited to only one opening with the desired powdered ring and elastic band. The covering can be discarded even with one use because it is economical in cost.

Accordingly, it is a principal object of the invention to provide an economical waterproof sheath covering for portions of a human body.

It is another object of the invention to provide an economical waterproof plastic sheath covering having external elastic bands at its open ends for a body part requiring a bandage or a cast.

It is a further object of the invention to provide a waterproof covering having an internally powdered region adjacent the external elastic band capable of forming a sealing gel upon wetting.

Still another object of the invention is to provide an internal wicking material layer between the waterproof covering and the powdered region.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a disposable, water resistant surgical forearm and hand cover over a cast having an internal powder ring at the forearm end (hidden) capable of gelling according to the present invention.

FIG. 2 is a cross-sectional view of a portion of the FIG. 1 cover illustrating the various critical parts of the cover.

FIG. 3 is an environmental, perspective side view of the invention on an injured foot and cast (hidden) as an illustration of the inventive cover with only one opening.

FIG. 4 is an environmental perspective front view of a cover for a person's torso having four openings.

FIG. 5 is an environmental perspective front view of a cover for a person's chest having two openings and a short pants cover having three openings.

Similar reference characters denote corresponding features consistently throughout the attached drawings.

### DETAILED DESCRIPTION OF THE PREFERRED EMBODIMENTS

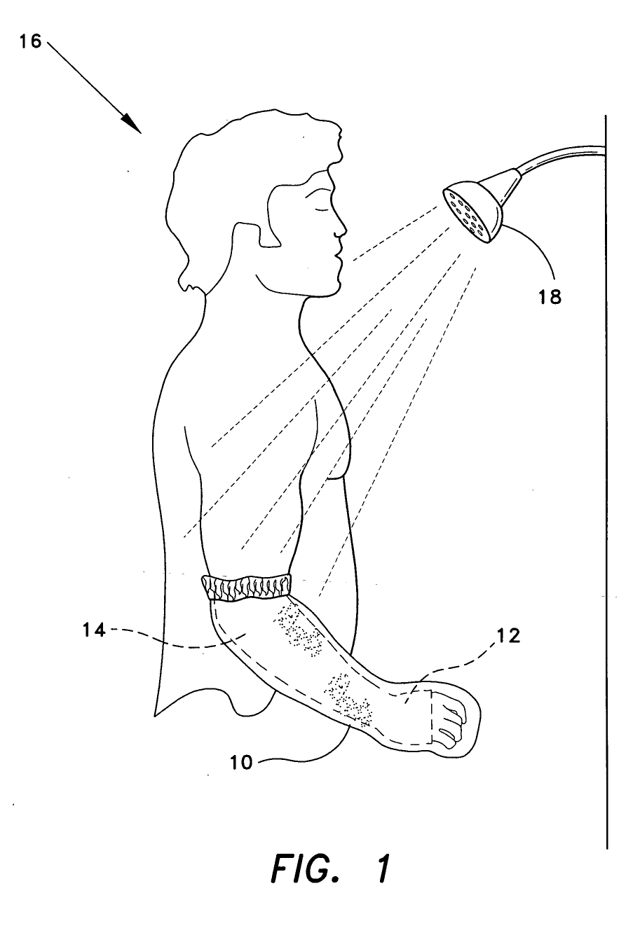

The present invention illustrated in FIG. 1 is directed to a flexible water resistant surgical bandage or cast covering 10 protecting either a surgical bandage or cast 12 (hidden) on the forearm and hand 14 of a man 16 showering under a shower nozzle 18. The covering 10 is made of a flexible, durable plastic composition having a film thickness typical of plastic utility bags. An elastic band 20 is sewn to the covering 10 by hemming the edge of the covering 10 over the band 20. The covering 10 can have any color or be transparent.

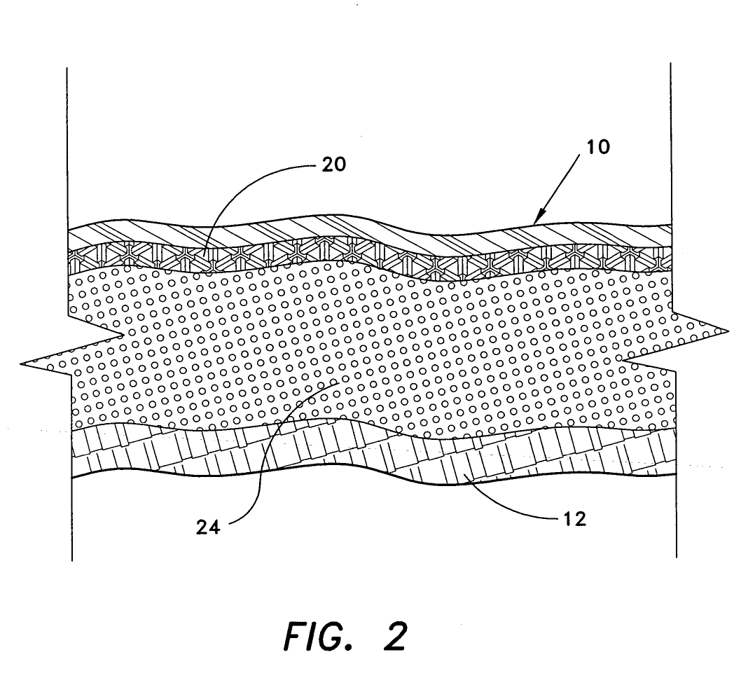

FIG. 2 illustrates the invention in a cross-sectional view to show only the plastic covering 10 attached to a wicking layer 22 made of fibrous cotton, and a layer 24 of the absorbent gel formed after contact with moisture over the plaster cast 12. The powder composition 26 forming the gel layer 24 is similar to that presently conventionally used in diapers.

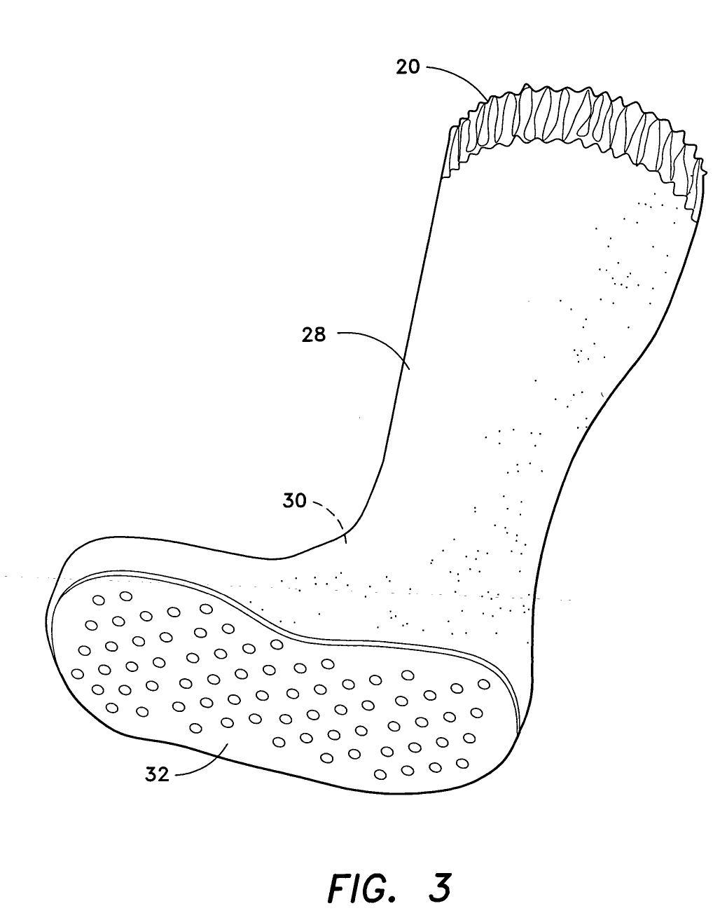

In FIG. 3, a cover 28 for a hidden foot cast 30 has an elastic band 20 on the single top opening with the internal compositions identical to those inside (hidden) as depicted in FIG. 2. A non-slip rubber sole 32 is adhesively added for protecting the cover 28.

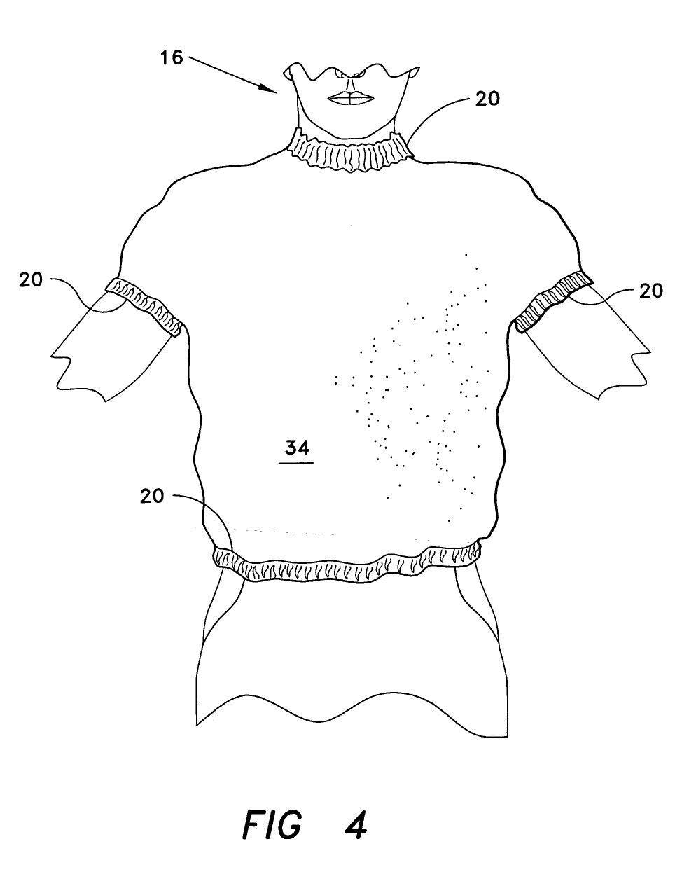

In FIG. 4, a full chest cover 34 similar to a pullover shirt, is illustrated with elastic bands 20 for each opening for the arms, neck and waist of the man 16. The internal elements are identical to those in the previous examples except for the waist opening that only has the external elastic band 24.

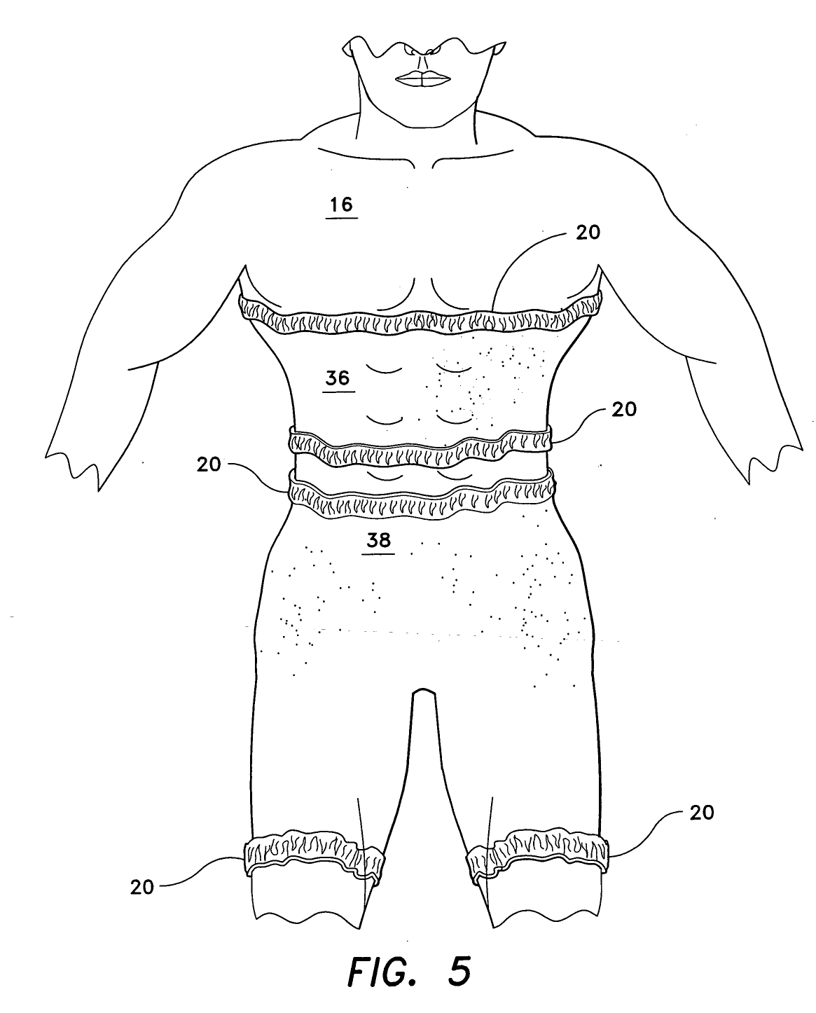

In FIG. 5, a partial chest cover 36 and a boxer shorts cover 38 combination are depicted with elastic bands 24 at each opening and having identical internal elements according to the present invention.

It is to be understood that the present invention is not limited to the embodiments described above, but encompasses any and all embodiments within the scope of the following claims.

We claim:

1. A disposable, water resistant surgical bandage or cast cover with internal powder rings at each open end, comprising:
a water resistant film cover in sleeve form;
an elastic band at either end encircling each opening; and
a powder ring capable of gelling when moistened and disposed adjacent each opening;
whereby the cover will prevent any water from entering while a wearer is taking a shower or a bath.

2. The cover according to claim 1, wherein the water resistant film cover is flexible plastic.

3. The cover according to claim 1, wherein the cover is adapted to cover a bandage.

4. The cover according to claim 1, wherein the cover is adapted to cover a cast.

5. The cover according to claim 1, wherein the cover is adapted to cover a body portion selected from the group consisting of the head, an arm, a chest, waist, a leg, and a foot.

6. The cover according to claim 5, wherein the cover is for a head.

7. The cover according to claim 5, wherein the cover is for an arm.

8. The cover according to claim 5, wherein the cover is for a chest.

9. The cover according to claim 5, wherein the cover is for a waist.

10. The cover according to claim 5, wherein the cover is for a leg.

11. The cover according to claim 5, wherein the cover is for a foot encased in a cast.
